(19) United States
(12) Patent Application Publication
(10) Pub. No.: US 2025/0041192 A1
(43) Pub. Date: Feb. 6, 2025

(54) IMPROVEMENTS IN OR RELATING TO ORGANIC COMPOUNDS

(71) Applicant: GIVAUDAN SA, VERNIER (CH)

(72) Inventor: Matthew Peter BROOKS, Ashford, Kent (GB)

(21) Appl. No.: 18/718,702

(22) PCT Filed: Dec. 15, 2022
(86) PCT No.: PCT/EP2022/086105

§ 371 Date: Jun. 11, 2024

(30) Foreign Application Priority Data
Dec. 15, 2021 (GB) 2118166.4

(51) Int. Cl. A61K 8/58; A61K 8/11; A61Q 13/00; A61Q 15/00; C11D 1/74; C11D 3/00; C11D 3/20; C11D 3/50; C11D 17/00

(52) U.S. Cl. CPC A61K 8/58; A61K 8/11; A61Q 13/00; A61Q 15/00; C11D 1/74; C11D 3/0015; C11D 3/0068; C11D 3/2079; C11D 3/505; C11D 17/0013; C11D 17/0039

(57) ABSTRACT

The present invention relates to a malodour reduction composition comprising zinc neodecanoate adapted to be delivered from an aqueous medium to a surface containing a source of malodour and to deliver a sensorially perceptible malodour reduction effect to the surface.

### FIELD OF THE INVENTION

The present invention is concerned with a malodour reduction composition comprising a zinc carboxylate, and to methods of suppressing or eliminating malodours by delivering said malodour reducing composition from an aqueous medium to a surface containing a source of malodour.

### BACKGROUND OF THE INVENTION

Human olfaction is complex, and how malodours are perceived can be influenced by many factors. However, the most important factor in sensing, and indeed eliminating malodours is the vapour phase concentration of the malodorous molecules. Suppression or elimination of malodour can be achieved by the use of malodour-counteracting agents that chemically modify malodorous molecules to create non-odorous reaction products; or by using malodour-counteracting agents that have anti-microbial activity to exert an effect on microbes that may act as precursors in the development of malodour. In either case, successful intervention usually relies upon the delivery of sufficiently high dosages of malodour-counteracting agents to a surface containing the sources of malodour, and retaining them on the surface for a time period sufficient to enable them to exert a malodour counteracting effect.

Zinc salts, including certain zinc carboxylates, are known to reduce or inhibit malodour, both by chemical and anti-bacterial means. Zinc neodecanoate, in particular, has been described as being useful as a malodour reducing active agent in aerosol antiperspirant compositions (WO2018/087147). Its excellent odour elimination benefits in aerosol antiperspirant compositions are at least comparable with, if not more effective than, the well-known aluminium-based antiperspirant actives, and may even be employed as a replacer for aluminium chlorohydrate in aerosol antiperspirant compositions (WO2018/087148). However, in both of these cases, zinc neodecanoate was employed in anhydrous formulations sprayed directly onto a surface containing a malodour source. In such cases in which high concentrations of zinc neodecanoate can be directly applied to a surface in need of treatment, one can anticipate high efficacy in terms of odour elimination.

Although zinc neodecanoate has proven to be an effective malodour counteracting agent when delivered from anhydrous formulations, particularly when directly sprayed onto a surface to be treated, its wider applicability into other product formats, and particularly aqueous product formats has been hampered by its very poor solubility or dispersibility in aqueous product formats. The difficulty of formulating zinc neodecanoate in aqueous formats, or in formats that are intended to be mixed with water in high dilutions when used by consumers, means that it has not been possible to create sensorially perceivable malodour reduction effects in such formats and explains why despite its effectiveness in the anhydrous aerosol deodorant format, it has not been exploited in consumer products including roll-on deodorants, hand dish wash products, surface cleaners, and fabric care products including but not limited to liquid and powder laundry detergents, fabric conditioners, fabric refreshers, and scent boosters.

### SUMMARY OF THE INVENTION

It is the object of the present invention to address the prior art and its attendant problems as they relate to the efficacy of malodour reducing technology for use in consumer products, and to provide a malodour reducing composition that can be delivered from an aqueous medium to a surface containing a source of malodour and deliver a perceivable sensorial benefit. Further objectives of the invention relate to methods of making and using said malodour reducing compositions.

Accordingly, the invention provides in a first aspect a malodour reducing composition comprising a zinc carboxylate and one or more ingredients selected from a perfume ingredient, a perfumery solvent, a surfactant, or mixtures thereof, wherein the composition is adapted to be dispersed in an aqueous medium.

In a second aspect, the invention provides a method of delivering a malodour counteracting effect to a surface containing a source of malodour, said method comprising the step of delivering a malodour reducing composition containing a zinc carboxylate to the surface from an aqueous medium.

In a third aspect, the invention provides a consumer product, comprising the malodour reducing composition defined herein.

The details, examples and preferences provided in relation to any one or more of the stated aspects or embodiments of the present invention will be further described herein and apply equally to all aspects and embodiments of the present invention. Any combination of embodiments, examples and preferences described herein in all possible variations thereof are encompassed by the present invention unless otherwise indicated herein, or otherwise clearly contradicted by context.

### DESCRIPTION OF THE FIGURES

FIG. 1. Perceived malodour intensity-results illustrating example 6 A

FIG. 2. Perceived malodour intensity of post-dosed refresher spray formulation on worn T-shirt inserts-results illustrating example 6 C

FIG. 3. Perceived intensity of malodour or fragrance-results illustrating example 6 D

### DETAILED DESCRIPTION OF THE INVENTION

The applicant has discovered in a surprising manner that despite the poor water solubility or dispersibility of zinc carboxylates, and in particular zinc neodecanoate, mixing or combining a zinc carboxylate in a selective manner with perfume ingredients, perfumery solvents, surfactants or technologies as more fully described herein, one can create malodour reducing compositions that can deliver a sufficient concentration of zinc carboxylate to a surface containing a source of malodour, even when delivered from highly dilute aqueous media, to deliver perceivable malodour reducing performance. The performance can be explained by the composition being adapted to deliver a high concentration of zinc carboxylate onto a surface to be treated. Furthermore, the applicant also surprisingly found that by carefully selecting the carboxylate ligand as described more fully herein, the zinc carboxylate can exhibit excellent film-forming properties on a surface and resist rinse-off.

In the first aspect of the invention the zinc carboxylate useful in the malodour reducing composition is selected on the basis of its ability to exert an effective malodour reducing effect on a surface containing a source of a malodour, when delivered from an aqueous medium. Relevant considerations in the selection include the solubility of the carboxylate in an oil phase containing one or more of the perfumery solvent, perfume ingredient, surfactants or mixtures thereof. The higher the carboxylate's solubility in an oil phase containing these ingredients, the higher can be its concentration in the malodour reducing composition. Other considerations include the ability of the carboxylate to disperse in an aqueous medium and deposit uniformly from it onto a surface to be treated; its ability to adhere to the surface once deposited; as well as its volatility. Carboxylates that deposit well, which are adherent to the surface and which do not evaporate quickly from the surface once deposited will, self-evidently, be able to exert a longer and more effective malodour reducing effect. Another important consideration is the odour of the carboxylate. For obvious reasons, it is undesirable if a malodour reducing compositions contains an active ingredient that is itself a source of malodour.

Zinc carboxylates having a carbon chain length of 1 to 3 carbon atoms are very water soluble and not compatible in perfumery solvents and perfume ingredients, and are not preferred as such. Those zinc carboxylates having a carbon chain length of 1 to 7 carbon atoms tend to have a pungent or even unpleasant odour and are likewise not preferred. However, zinc carboxylates, wherein the carboxylate group has between 8 and 12 carbon atoms are preferred as they have optimum malodour reduction performance, have a low volatility and also have low or no odour. Most preferred of the zinc carboxylates is zinc neodecanoate. Zinc neodecanoate and methods for its preparation, for example from zinc oxide and the corresponding carboxylic acid, are well known in the art.

A malodour reducing composition is considered to be adapted to be dispersed in an aqueous medium for the purpose of the present invention if the composition, by means of the use of particular mixtures of perfumery ingredients, solvents or surfactants, and/or the use of certain technologies, such as encapsulation technologies, will readily disperse in an aqueous medium when the composition is incorporated into an aqueous consumer product composition, or when it is diluted in water in use by a consumer.

In particular embodiments of the present invention, the malodour reducing composition is adapted to be dispersed in an aqueous medium by providing the zinc carboxylate in an encapsulated form.

Encapsulated zinc carboxylate can be presented in the form of a plurality of microparticles. Microparticles are small particles, having diameters ranging generally from about 1 to 1000 microns. Microparticles may have variety of structures ranging from single-core to multi-cores, and can comprise single or multilayer shells. The core material can comprise a zinc carboxylate and any other desired ingredient, such as one or more perfume ingredients or perfumery solvents, or surfactants. The microparticle core can be loaded with any level of zinc carboxylate, up to 100 wt %. More particularly however, the core can contain zinc carboxylate in admixture with one or more perfume ingredient, perfumery solvent, surfactant or mixtures thereof, in which case, the core may contain upwards of about 10, 20, 30, 40, 50, 60 or 70 wt % of zinc carboxylate.

The microparticles may be presented in the form of an aqueous slurry in which the microparticles are suspended. The aqueous medium can contain suspending agents, preservatives and any other excipients commonly known in the art to ensure that the microparticles are incorporated stably into the slurry. If desired, the aqueous slurries can be dehydrated, for example by spray drying, to reduce the level of moisture present and to present the microparticles in a powdered format. Methods of forming microparticle slurries or dehydrated microparticles in powder format are well known in the art, and examples of such methods are disclosed in the references cited herein below, all of which are incorporated into the specification by reference.

The shell material used in the microparticles can vary depending on the type of consumer product into which they will be incorporated.

Conventionally, shell materials can comprise thermosetting polymers, such as melamine formaldehyde, melamine urea formaldehyde, polyurea, polyurethane and polyacrylates. Such thermosetting polymers shells can be formed as shells by known polymerization methods, such as interfacial polymerization around droplets of core material. Examples of core shell microcapsules formed from thermosetting polymers that are useful in the preparation of encapsulated zinc carboxylates are disclosed in WO2004/016234; WO2006/056093; WO2007/137441; WO2008/098387; WO2009/100553; WO2017/001672; WO2018/197266; WO2016/207180; WO2018/149775; WO2011/161229; WO2013/092958; WO2016/071151; WO2016/071150; WO2016/071149; WO2017/085105; WO2014/064252; WO2014/064255; and WO2014/032290, all of which documents are herein incorporated by reference.

Another commonplace method of forming core-shell microparticles useful in the present invention is coacervation. Coacervation is a well-known method of forming capsules, whereby colloids are caused to form and harden around droplets of core material. So-called simple coacervation uses a single hydrocolloid, whereas complex coacervation uses two hydrocolloids. Coacervate microparticles useful in the present invention are disclosed in WO2015/150370; and WO2013/068581, which documents are herein incorporated by reference.

Still further, encapsulated compositions can be formed by spray drying an emulsion comprising a zinc carboxylate and shell forming polymers, such as starches, celluloses and the like. Examples of such microparticles, and methods of preparing them, are disclosed in WO2020/149192; WO2015/189296; and WO2020/201258 all of which documents are herein incorporated by reference.

More recently, the perfume industry has moved in the direction of biodegradable microparticles that are free, or substantially free of microplastics, and such microparticles are useful to encapsulate zinc carboxylates in accordance with the present invention. Biodegradable microparticles can be formed by a process of coacervation or complex coacervation using various proteins and/or polysaccharides. Examples of biodegradable microparticles useful in the present invention are disclosed in WO2020/233887, which is herein incorporated by reference.

An encapsulated form of zinc carboxylate can also be presented in the form of a plurality of pastilles or prills, which can be formed when the zinc carboxylate is dissolved or dispersed in a water-soluble matrix material and the resultant mixture is shaped, for example by extrudation and cutting. Alternatively, molten droplets of matrix material and zinc carboxylate can be dropped into a suitable liquid bath to harden the droplets and form prills, which can be collected by filtration.

The water soluble matrix can consist of a variety of materials useful in the preparation of scent booster compositions, as are generally well known in the art. An example of a suitable matrix material would be a mixture of polyethylene glycol, a filler material, and optionally a clay or a salt or mixtures thereof. Such compositions are described in US2017/226690, which is herein incorporate by reference. The zinc carboxylate, and optionally a perfume ingredient, perfumery solvent and a surfactant can be incorporated into the matrix material freely, or in an encapsulated form, as more fully described above. Encapsulated forms of zinc carboxylate described hereinabove can also be incorporated into such water-soluble matrices.

In other embodiments of the present invention, the malodour reducing composition may be adapted to disperse in aqueous media, by dissolving or dispersing the zinc carboxylate in an oil phase comprising a surfactant and optionally at least one perfumery ingredient and or perfumery solvent, or mixtures thereof.

Whereas the zinc carboxylates are to some extent soluble or miscible in perfumery ingredients or perfumery solvents, they are practically insoluble in water. Accordingly, in order to be able to disperse an effective concentration of a zinc carboxylate in a water-based consumer product, or in a consumer product that in use is intended to be diluted in water, it is necessary to present the zinc carboxylate in admixture with a surfactant.

Preferred surfactants are non-ionic surfactants. Suitable non-ionic surfactant include any of those that are commonly employed in consumer products including but not limited to roll-on deodorants, hand dish wash, surface cleaners, deodorants, and fabric care products, including but not limited to liquid and powder laundry detergents, fabric conditioners, fabric refreshers, and scent boosters. Particularly preferred non-ionic surfactants include ethoxylated fatty acids and particular those having a chain length the same as or similar to that of the zinc carboxylate employed. Examples of particularly preferred non-ionic surfactants are Lutensol TO10 or Synperonic 13/9.

In accordance with the second aspect of the invention, the malodour reducing composition is delivered from an aqueous medium to a surface containing a source of malodour.

In particular embodiments of the present invention, the malodour reducing composition described hereinabove is in a finished form. That is, the malodour reducing composition is a finished article of manufacture that is intended to be incorporated as such into a consumer product base.

For example, in a particular embodiment, the malodour reducing composition in the form of an aqueous slurry comprising a plurality of microparticles can be mixed into an aqueous consumer product base to form a consumer product.

Similarly, the malodour reducing composition in the form of a dry powder comprising a plurality of zinc carboxylate-containing microparticles, can be mixed into a dry powder consumer product base to form a consumer product in dry powder form. An example of such a consumer product would be a dry powder detergent, which can be dissolved or dispersed in an aqueous medium during use.

In other embodiments of the invention the malodour reducing composition is not a finished article of manufacture as such, but each ingredient of the composition may be added separately to a consumer product base in sequential or simultaneous steps to form the malodour reducing composition in situ. For example, an oil phase comprising zinc carboxylate dissolved in one or more perfume ingredients or perfumery solvents, or mixtures thereof, can be added to a consumer product base which already contains a non-ionic surfactant, thereby to form a finished malodour reducing composition in-situ at the same time as the consumer product is formed in essentially the same manufacturing operation. Alternatively, a mixture of zinc carboxylate and a non-ionic surfactant can be added to a consumer product base already containing perfume ingredients.

Zinc carboxylates can be delivered from an aqueous medium to all manner of surfaces that contain a source of malodour by means of the present invention. Surfaces include human or animal skin or hair, or inanimate surfaces including all manner of household surfaces such as hard surfaces, floors, bathroom and toilets, dishes, cutlery and other kitchen utensils, fabrics and the like. Malodours found on such surfaces include malodours from food, body odours, and odours from the waste of humans and animals.

Sources of malodour include, but are not limited to personal malodours such as axillary sweat, foot malodour, feminine (vaginal) malodour, scalp/hair malodour, urine malodour, food waste malodour, indoor air malodour, malodour from mould and mildew, and laundry malodour.

In the third aspect of the invention the consumer products into which a malodour reducing composition can be added include, but are not limited to, carpet spray, fabric spray, all-purpose cleaners, bathroom cleaners, kitchen cleaners, floor cleaners, hand dish wash cleaners, diapers, feminine hygiene products, cat litter, roll-on deodorants, roll-on anti-perspirants, liquid soap, bar soap, body wash, detergent bars, detergent pastes/creams, detergent powder tablets, liquid detergents, liquid detergent capsules, detergent powders, fabric fresheners, scent boosters, ironing water, and liquid softeners.

The malodour reducing composition can be added to the aforementioned consumer products at levels such as to deliver an effective amount of zinc carboxylate onto a surface to exert an effective malodour reducing effect. In particular embodiments, the dosage of the malodour reducing composition is such that the total amount of zinc carboxylate in the consumer product is about 10 wt % or less, more particularly 9, 8, 7, 6, 5, 4, 3, 2, 1, 0.5, 0.4, 0.3, 0.2 or 0.1 wt % or less, based on the total weight of the consumer product.

The invention will be further explained and illustrated with reference to the following non-limiting examples.

### Example 1

### (Synthesis of a Melamine Urea Formaldehyde Capsules Containing the Malodour Reducing Composition)

The malodour reducing composition (1 wt % perfume, 19 wt % isopropyl myristate, and 80 wt % zinc neodecanoate) is encapsulated in melamine urea formaldehyde to form 1 Kg of slurry in accordance with the following method:

A reactor set to a temperature of 20° C. and is charged with deionised water (600 g). Resorcinol as cross-linker (10 g); positively charged polymeric colloid stabilizer (2 g) and melamine formaldehyde pre-condensate (Luracoll SD) (5 g). The stirring speed is set to 400 rpm. At this stage, 360 g of the malodour reducing composition (50 wt % in medium chain triglyceride) is added and dispersed in the water phase.

Polymerisation is undertaken in the following manner: Formic acid (10%) is added to the slurry until the pH reaches 4, and the slurry is stirred for 1 hour at 35° C. Thereafter, the reactor temperature is increased to 90° C. over a period of 1 hour.

Finally, the slurry is cooled, and the pH is adjusted with the addition of ammonia (1 g) until it falls into the range of 3-5. Deionised water is finally added to bring the volume of the slurry to 1 L. The resultant slurry of encapsulated composition is discharged from the reactor.

The malodour reducing composition was successfully encapsulated in the slurry.

### Example 2

### (Synthesis of a Starch Capsule Containing the Malodour Reducing Composition)

Tap water (55.0 g) was weighed into a stainless steel beaker. Starch sodium octenyl succinate E1450 (18.7 g), starch modified Hi-Cap 100 (2.2 g) and maltodextrin Glucidex IT-19 (5.3 g) were subsequently weighed into the same beaker. The resulting mixture was first manually stirred with a stainless steel rod and then homogenized with an IKA T25 Ultra-Turrax Homogenizer at 13,500 rpm to obtain a homogeneous solution. To this resulting mixture was added the malodour reducing composition of Example 1 (17.8 g). High shear mixing was then carried out for 20-30 min at 22,000-24,000 rpm using the same Homogenizer to produce an emulsion. Dynamic light scattering was used to ensure the droplet size was between 0.5 and 2 microns. The emulsion was subjected to spray drying using a LabPlant SD-06 Spray Dryer. The spray drying process parameters were as follows: Inlet Temperature: 190° C.; Outlet Temperature: 90° C.; peristaltic pump speed: 485 mL/h; and air flow rate: 3.7 m/s.

The resulting spray dried powder was mixed with silicon dioxide Aerosil 200 (0.5 g) in a closed mixing vessel.

The malodour reducing composition was successfully encapsulated in the dried powder.

### Example 3

### (Synthesis of a Scent Booster Composition Containing the Malodour Reducing Composition)

A scent booster composition was prepared by blending fumed silica (Aerosil 200)@5 wt %, CMC (Blanose)@5 wt %, and sodium chloride @ 80 wt. 10 wt % and the malodour reducing composition of Example 1 was added on top of the scent booster mixture and the whole was blended until a free-flowing powder is achieved.

(AEROSIL is a fumed silica (trade mark of Degussa); “Blanose” is carboxymethyl cellulose (trade mark of Hercules))

### Example 4

(Deposition of Zinc Neodecanoate from the Scent Booster Formulation of Example 3 on Fabric)

### Use of a Metallochromic Indicator to Determine Deposition on Fabric

Eriochrome Black T is a metallochromic indicator that can be used for complexometric titration with various metal ions. It is typically used with a pH10 buffer, whereupon the solution changes from a blue colour to red when forming a complex with a metal ion, such as zinc. The proportion of complexed Eriochrome Black T will determine the shade of the indicator solution. The presence of any uncomplexed indicator will remain blue, which can mask the red coloured complexed form. Therefore, it is important to use as little of the indicator as possible to make the test as sensitive as possible. However, this needs to be carefully balanced with using enough indicator to perceive any colour.

A series of solutions of zinc neodecanoate in ethanol were prepared (from 10% to 0.00001% in logarithmic steps). A further series of vials were prepared with 2 ml of pH 9.41 buffer solution and 10 μL of Eriochrome Black T solution (1% in ethanol). 10 μL of the zinc neodecanoate solutions were added to the vials with the Eriochrome Black T solutions. The colours of the resulting solutions demonstrate that zinc neodecanoate can be detected at concentrations of 0.1% and higher, with 0.01% producing a weakly positive result.

### Simulated Wash Cycle Test

10 wt % of a malodour reducing composition (zinc neodecanoate@10 wt % in Dowanol TPM) is added to the scent booster composition described in Example 3, and mixed thoroughly. A sample only using Dowanol TPM and the scent booster composition was also prepared. 150 mg of each scent booster composition was added to 50 ml of distilled water in a 60 g glass bottle. Four small pieces of terry towelling (totalling 14 cm²) were added to each bottle and placed on a roller for one hour.

The indicator solution was prepared by adding buffer solution (1 mL, pH 9.21) and Eriochrome Black T solution (1% in ethanol) into a clear glass vials (7.5 mL). One of the pieces of terry towelling from each bottle was added to separate vials containing the indicator solution. The vials were agitated and the resulting colour was recorded.

Further indicator (10 μL of 1% Eriochrome in ethanol) was added to each vial and the colours became more differentiated

The remaining pieces of terry towelling were transferred to a fresh clear glass bottle (60 mL) containing distilled water (50 mL). The bottles were placed on a roller for a further hour. A piece of terry towelling was removed from each bottle and tested with the indicator solution as before (10 μL of 1% Eriochrome in ethanol, in 1 ml of pH9.21 buffer solution). It is still possible to differentiate the colours of the resulting solutions. This demonstrates that zinc neodecanoate is depositing onto the fabric and remaining there after rinsing.

The two remaining pieces of cloth were air dried overnight and tested using the same procedure as before. The cloth washed along with the scent booster containing zinc neodecanoate was still positive for the presence of zinc This experiment demonstrated that zinc neodecanoate can deposit onto fabrics and remain there after rinsing and drying.

### Example 5

### (Demonstration of Malodour Reduction Performance on Fabric-Trigger Spray Formulation)

A clear and colourless model trigger spray formulation was prepared using zinc neodecanoate (0.75 wt %), Dowanol TPM as solvent (0.25 wt %), lutensol TO 10 (9 wt %) and water (90 wt %). A control was prepared by replacing Dowanol TPM and zinc neodecanoate with diethyl phthalate (1 wt %).

T-shirt inserts were pre-treated with the model formulation and the control. The inserts were allowed to dry and were placed into the underarm of t-shirts, which were then worn by a panel of volunteers for a whole day, without using fragranced products. Thereafter, the t-shirt inserts were olfactively assessed by a trained sensory panel for malodour intensity.

It was found that those inserts treated with the model trigger spray formulation exhibited significantly reduced malodour intensity compared with those inserts treated with the control formulation.

### Example 6

### (Demonstration of Malodour Reduction Performance on Fabric-Laundry Tests)

### A. Odour Elimination on Terry Towelling

### Preparation

Three different test compositions (1-3) were prepared according to the composition given in table 1. The samples NU67, MO48 and QC29 (1000 μL) are applied to the centre of separate pieces of cloth (70 mm×70 mm of terry towelling squares) together with 250 μl of 0.006% 3-mercapto-3-methyl butan-1-ol (MMB) in Dowanol TPM as malodour. Samples ED71 and WC14 are applied to the centre of separate pieces of cloth (70 mm×70 mm of terry towelling squares) without any malodour as control. Labels were attached to the top right of each piece of fabric.

### Results:

The pieces of fabric were assessed by a trained panel of 15 assessors for malodour intensity using a 0-100 scale with the control set at 70, and 1-minute gap between assessments. The samples were assessed in a randomised order (randomisation was produced using Design Express). The average intensity rating for the hidden control (NU67) was 63. The panel reliability was very good (G=0.98, phi=0.98, inter-rater reliability=0.76).

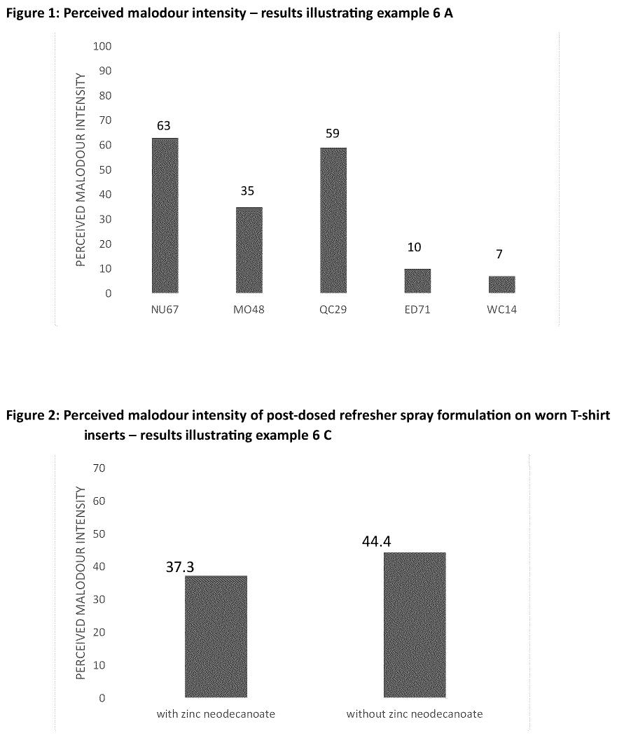

TABLE 1
Samples and results (also FIG. 1)

| | | | Malodour | Perceived Malodour |
|---|---|---|---|---|
| Sample | Test | Composition | Applied | intensity |
| NU67 | 1 | 1% Dowanol TPM, | Yes | 63 |
| | | 9% Synperonic 13/9, | | |
| | | 90% water | | |
| MO48 | 2 | 0.25% Dowanol TPM, | Yes | 35 |
| | | 0.75% Zinc decanoate, | | |
| | | 9% Synperonic 13/9, | | |
| | | 90% water | | |
| QC29 | 3 | 0.25% Dowanol TPM, | Yes | 59 |
| | | 0.75% TegoSorb Conc. 50, | | |
| | | 9% Synperonic 13/9 | | |
| | | 90% water | | |
| ED71 | 1 | 1% Dowanol TPM, | No | 10 |
| | | 9% Synperonic 13/9, | | |
| | | 90% water | | |
| WC14 | 2 | 0.25% Dowanol TPM, 0.75% | No | 7 |
| | | Zinc decanoate, | | |
| | | 9% Synperonic 13/9, | | |
| | | 90% water | | |

### B. Odour Elimination by Application of Refresher Spray-Pre-Dosed on T-Shirt Inserts

### Methodology—Test Formulations

Two refresher sprays were formulated, one with zinc neodecanoate, the other one without it. They were prepared by mixing each ingredient in the order presented in table 2, from top to bottom.

TABLE 2
Composition of fabric refresher spray formulations

| | Amount (%) | Amount (%) |
|---|---|---|
| Ingredient | in +TECH | in W/O |
| Zinc neodecanoate | 0.75 | — |
| Dowanol TPM | 0.25 | 1.00 |
| Synperonic 13/7 | 9.00 | 9.00 |
| Water | 90.00 | 90.00 |

### Methodology—Insert Preparation

The fabric inserts are pre-washed, de-sized, labelled, had poppers sewn-in and hung on a laundry drying rail. The fabric refresher formulations are applied to each T-shirt insert: 2 sprays of the formulation followed by 2 further sprays 5 minutes later. The inserts are then allowed to dry before attaching to the T-shirts, randomising which side had the technology present (+TECH) and which not (W/O) as shown in table 3.

TABLE 3
Order used for location of each refresher spray formulation

| T-Shirt No. | Left Insert | Right Insert |
|---|---|---|
| 1 | +TECH | W/O |
| 2 | W/O | +TECH |
| 3 | +TECH | W/O |
| 4 | W/O | +TECH |
| 5 | +TECH | W/O |
| 6 | W/O | +TECH |
| 7 | +TECH | W/O |
| 8 | W/O | +TECH |
| 9 | +TECH | W/O |
| 10 | W/O | +TECH |
| 11 | +TECH | W/O |
| 12 | W/O | +TECH |
| 13 | +TECH | W/O |
| 14 | W/O | +TECH |
| 15 | +TECH | W/O |
| 16 | W/O | +TECH |
| 17 | +TECH | W/O |
| 18 | W/O | +TECH |
| 19 | +TECH | W/O |
| 20 | W/O | +TECH |
| 21 | +TECH | W/O |
| 22 | W/O | +TECH |
| 23 | +TECH | W/O |
| 24 | W/O | +TECH |
| 25 | +TECH | W/O |

The T-shirts are then supplied to volunteers, who wear them for a working day without use of any fragranced products or deodorant. The T-shirts are then returned for assessment the following day.

### Methodology—Assessment of Trained Sensory Panel

The inserts are removed from the T-shirts and arranged around two separate tables (one for left inserts, the other for the right inserts). The trained sensory panel (n=16) assess each insert individually for malodour intensity using a 0-100 free scoring scale in a randomised order defined by the assessment sheet.

### Results

23 T-shirts were returned (1 of which had not been worn, but was included as a hidden control) and were assessed by the trained sensory panel. It was noted that several of the inserts smelled of fragrance and were later removed from the results.

The reliability of the panel assessments was checked using the Panel Reliability Program (v2-8) and no assessors needed to be excluded (G=0.93, phi=0.91, inter-rater reliability=0.47).

The malodour intensity of the pairs of T-shirt inserts with LS means below 20 were considered too low and removed. The remaining dataset was analysed by ANOVA.

The geometric mean malodour intensity was calculated for each insert, along with the standard error and difference between left and right. Perceived malodour intensity was measured and the overall results are that, with a value of 42.5, the intensity of the pre-dosed T-shirt inserts with zinc neodecanoate was lower than the intensity of the T-shirt inserts without the zinc neodecanoate having a value of 45.2.

### C. Odour Elimination by Application of Refresher Spray-Post-Dosed on Worn T-Shirt Inserts

### Methodology—Test Formulations

Two refresher sprays were formulated, one with zinc neodecanoate, the other one without it. They were prepared by mixing each ingredient in the order presented in table 4, from top to bottom.

TABLE 4
Composition of fabric refresher spray formulations

| | Amount (%) | Amount (%) in |
|---|---|---|
| Ingredient | in +TECH | W/O |
| Zinc neodecanoate | 0.75 | — |
| Dowanol TPM | 0.25 | 1.00 |
| Synperonic 13/7 | 9.00 | 9.00 |
| Water | 90.00 | 90.00 |

### Methodology—Insert Preparation (Before Wear)

The fabric inserts are pre-washed, de-sized, labelled, had poppers sewn-in and attached to the under-arm area of white T-shirts (also pre-washed and de-sized). The T-shirts are then supplied to volunteers who wear them for a working day without use of any fragranced products or deodorant. The T-shirts are then returned for further treatment and assessment.

### Methodology—Insert Preparation (After Wear)

The fabric inserts were pre-screened by a trained panel of three internal sensory assessors to ensure that there is a perceivable amount of malodour but no fragrance present on the inserts. The fabric refresher formulations are applied to the remaining inserts: approximately 4 pumps of the selected fabric refresher. A different fabric refresher is applied to either the left or right insert. The inserts are then allowed to dry before attaching to the T-shirts, randomising which side had the technology present (+TECH) and which not (W/O) as shown in table 5.

TABLE 5
Order used for location of each refresher spray formulation

| T-Shirt No. | Left Insert | Right Insert |
|---|---|---|
| 2 | W/O | +TECH |
| 3 | +TECH | W/O |
| 4 | W/O | +TECH |
| 5 | +TECH | W/O |
| 6 | W/O | +TECH |
| 7 | W/O | +TECH |
| 8 | +TECH | W/O |
| 9 | +TECH | W/O |
| 13 | +TECH | W/O |
| 14 | W/O | +TECH |
| 15 | W/O | +TECH |
| 16 | +TECH | W/O |
| 17 | +TECH | W/O |
| 18 | W/O | +TECH |
| 22 | W/O | +TECH |
| 24 | W/O | +TECH |
| 25 | +TECH | W/O |

### Methodology—Assessment of Trained Sensory Panel

The inserts are allowed to dry and then arranged in pairs for olfactive assessment. The trained sensory panel assesses each insert for malodour intensity using a 0-100 free scoring scale in a randomised order defined by the assessment sheet.

### Results

There were 17 pairs of T-shirt inserts assessed by the trained sensory panel (n=15).

The malodour intensity data provided by the trained internal sensory panel was checked using the Panel Reliability Program (v2-8). The results indicate that no assessors need to be excluded (G=0.93, phi=0.90, inter-rater reliability=0.48).

The malodour intensity of the pairs of T-shirt inserts with LS means below 20 were considered too low and removed. The remaining dataset was analysed by ANOVA.

The geometric mean malodour intensity was calculated for each insert, along with the standard error and difference between left and right. Perceived malodour intensity was measured and the overall results are that, with a value of 37.3, the intensity of the post-dosed T-shirt inserts with zinc neodecanoate was lower than the intensity of the T-shirt inserts without the zinc neodecanoate having a value of 44.4 (FIG. 2).

D. Fabric Conditioner with Socks

### Test Formulations

Two sets of fabric conditioners were formulated, one with zinc decanoate and the other one without it. The fragrance selected is a ‘typical’ fabric conditioner type fragrance.

TABLE 6
Composition of liquid fabric conditioner formulations (%)

| | Amount (%) | Amount (%) |
|---|---|---|
| Ingredient | With | Without |
| Zinc decanoate | 5 | — |
| distilled water | — | 5 |
| Fragrance | 1 | 1 |
| Fab Con Base | 94 | 94 |

### Methodology—Washing Socks

Sixty pairs of socks (30 male, 30 female) were separated and washed such that either the left or right were treated either with (+TECH) or without the technology (W/O) as shown in table 7. A standard wash cycle was used with un-fragranced detergent. The fabric conditioner (35 g) was placed directly into the drum at the appropriate part of the wash cycle.

TABLE 7
Sequence used to sort socks treated with
different fabric conditioner sample

| | Left | Right |
|---|---|---|
| Male Socks - Odd | +TECH | W/O |
| Male Socks - Even | W/O | +TECH |
| Female Socks - Odd | W/O | +TECH |
| Female Socks - Even | +TECH | W/O |

### Methodology—Wear and Self-Assessment

The socks were provided to volunteers (naive panellists) to be worn for a working day, then assessed for fragrance and malodour (0-10 fixed scale), and the preferred socks were selected.

Results 30 pairs of male socks and 29 pairs of female socks were returned along with the assessment sheets. There was no clear difference in preference between the socks washed using the fabric conditioner with zinc decanoate, compared to without (16 men with, 13 men without, 15 women with, 13 women without).

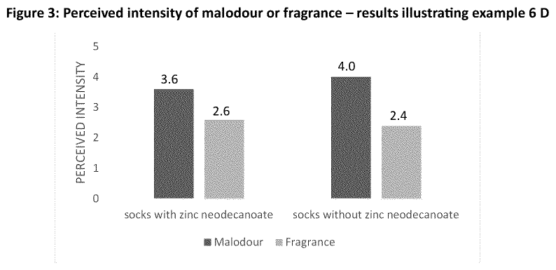

Several of the socks were self-assessed as not having any malodour and were removed from the data set. The resulting dataset was used to compare the fragrance intensity and malodour intensity of the samples with and without zinc decanoate present. It was found that there is an indicative benefit in malodour intensity, which however is not statistically significant. There is no difference in perceived fragrance intensity (FIG. 3).

1. A malodour reducing composition comprising a zinc carboxylate and one or more ingredients selected from a perfume ingredient, a perfumery solvent, a surfactant, or mixtures thereof, wherein the composition is configured to be dispersed in an aqueous medium.

2. The malodour reducing composition according to claim 1 wherein the zinc carboxylate is in encapsulated form.

3. The malodour reducing composition according to claim 2 in the form of an aqueous slurry of core shell microparticles, the core containing a zinc carboxylate.

4. The malodour reducing composition according to claim 1, wherein the zinc carboxylate is dissolved or dispersed in an oil phase comprising a surfactant and optionally at least one perfumery ingredient and/or perfumery solvent.

5. The malodour reducing composition according to claim 4, wherein the surfactant is a non-ionic surfactant.

6. The malodour reducing composition according to claim 4, wherein the surfactant is an ethoxylated fatty acid.

7. A consumer product comprising a malodour reducing composition according to claim 1.

8. The consumer product according to claim 7, selected from the group consisting of carpet spray, fabric spray, all-purpose cleaners, bathroom cleaners, kitchen cleaners, floor cleaners, hand dish wash cleaners, diapers, feminine hygiene products, cat litter, roll-on deodorants, roll-on anti-perspirants, liquid soap, bar soap, body wash, detergent bars, detergent pastes, detergent creams, detergent powder tablets, liquid detergents, liquid detergent capsules, detergent powders, fabric fresheners, scent boosters, ironing water, and liquid softeners, and combinations thereof.

9. A method of delivering a malodour counteracting effect to a surface containing a source of malodour, said method comprising the step of delivering a malodour reducing composition containing a zinc carboxylate to the surface from an aqueous medium.

10. The method according to claim 9, wherein the surface is selected from human or animal skin or hair, or inanimate surfaces.

11. The method according to claim 9, wherein the malodour is selected from a body odour, a scalp malodour, malodour from food waste or from the waste of humans or animals.

12. The malodour reducing composition according to claim 5, wherein the surfactant is an ethoxylated fatty acid.

13. The method according to claim 10, wherein the inanimate surfaces comprise household surfaces.

14. The method according to claim 13, wherein the household surfaces are selected from at least one of: hard surfaces, floors, bathroom and toilets, dishes, cutlery, kitchen utensils and fabrics.
